(19) United States
(12) Patent Application Publication
(10) Pub. No.: US 2008/0306454 A1
(43) Pub. Date: Dec. 11, 2008

(54) Apparatus And Method For Sterilization Of An Intravenous Catheter

(76) Inventor: Christopher F. Sikora, Elmhurst, IL (US)

Correspondence Address: Husch Blackwell Sanders, LLP;Welsh & Katz, 120 S RIVERSIDE PLAZA, 22ND FLOOR, CHICAGO, IL, 60606, US

(21) Appl. No.: 11/759,071

(22) Filed: Jun. 6, 2007

(51) Int. Cl. A61M 25/00; A61L 2/10

(52) U.S. Cl. 604/265; 422/24

(57) ABSTRACT

An intravenous port and method of sterilizing an intravenous port via an ultraviolet (UV) light source are provided. The port comprises a catheter capable of being inserted into a blood vessel and an access head having a signal receptor, such as an antenna, and ultraviolet light source such as for example a light emitting diode (LED). A transmitter is further provided for delivering an electrical signal from a power source to the UV light source. The light source is suitable for directing UV light of a predetermined wavelength through the lumen of the catheter in a manner that incapacitates microorganisms residing in the catheter. The UV light is transmitted through the lumen of the catheter through a sterile solution that can be inserted into the port.

### FIELD OF THE INVENTION

The present invention is directed to an apparatus and method for sterilization of an intravenous catheter and is particularly directed to a device and process which utilizes ultraviolet light in order to deactivate bacteria or other microorganisms within an intravenous long-dwelling catheter.

### BACKGROUND OF THE INVENTION

Often times, patients receiving medical treatment for a chronic health condition will require frequent or continuous administration of intravenous substances and will thus have a central venous access device or “central line” inserted into a major vein such as the jugular, subclavian or femoral vein through the chest, neck or groin area. Examples of such central lines include Hickman catheters, Broviac catheters, Hohn catheters or Port-a-Caths®. Such catheters are commonly used to repeatedly draw blood for laboratory assessments and to administer medications, intravenous fluids, blood products, and, at times, intravenous nutrition.

Although intravenous catheters have been shown to have numerous medical benefits, one of the more common risks associated with their use is the onset of catheter-related bacterial infections. While all catheters are known to deliver bacteria into the bloodstream, central venous catheters, because of their placement and longevity of use have been known to occasionally become contaminated with bacteria or other microorganisms in a manner which can cause serious infections, including for example Staphylococcus aureus and Staphylococcus epidermidis sepsis.

Some catheter-related infections arise from microorganisms entering the body at the insertion site of the catheter, while others arise from the microorganisms entering the body via the catheter itself. While a catheter is generally sterile when it is first inserted into a patient, it is typical for microorganisms to enter the catheter from the external environment through openings in the catheter. The microorganisms may then adhere to the interior surface of the catheter and colonize. An infection may occur in the patient when microorganisms from the catheter are transmitted into the body of the patient via the flow of fluids into the patient. In addition to seriously jeopardizing the health of an already weak patient, such infections can compromise the catheter, require its surgical removal and delay medical treatment. In many instances, infected patients must be treated with significant dosages of antibiotics in order to eradicate the bacteria and prevent the continued spread of the microorganism within the patient's body.

To prevent infection, some intravenous catheters are coated or impregnated with antibiotics or antiseptics such as silver sulfadiazine. Catheters may also be flushed with a sterile solution such as saline in order to disinfect the interior of the catheter and prevent infection. These existing methods however pose several problems and limitations. Specifically, antiseptics such as silver sulfadiazine are typically slightly acidic compositions that can cause a patient to experience an uncomfortable burning sensation at the catheter's insertion point or can cause damage or irritation to the skin around the area of the catheter. Examples of such damage or irritation can include skin discoloration, a severe rash or redness and even damage to an entire areas of skin cells. In addition, coating a catheter with antibiotics for prophylactic purposes raises concerns about the potential emergence of antibiotic-resistant strains. Finally, flushing the line of a catheter with saline or other types of sterile solutions may not be by itself effective in eliminating many microbes that are particularly well-adhered to the interior of the catheter.

These types of sterilization methods also typically require repeated manual application or attention in order to be effective for long term catheter use. As such repeated application or attention can be time consuming, expensive and easily and often overlooked in a healthcare setting; a need exists for a sterilization device and technique for using the device that is automated, inexpensive and requires less manual attention. Accordingly, an improved, more reliable and more automated method for sterilizing an intravenous catheter is necessary.

### SUMMARY OF THE INVENTION

Ultraviolet Germicidal Irradiation, or “UVGI” is a sterilization method that uses ultraviolet (“UV”) light at a sufficiently short wavelength to break down micro-organisms. It is used in a variety of applications, such as food, air and water purification or medical sterilization, and has generally been shown to be a highly effective method of destroying many types of microorganisms. At certain wavelengths UV light is mutagenic to bacteria, viruses and other microorganisms. At a wavelength between 100-280 nm, UV light, generally referred to as UVC at this particular wavelength, can cause damage to the nucleic acid of microorganisms by forming covalent bonds between certain adjacent bases of the microorganism's DNA. The formation of such bonds prevents the DNA from unzipping for replication and the organism is thus unable to reproduce. In fact, once covalent bonds are formed between adjacent bases of DNA, the organism will be destroyed if it tries to replicate. Since many types of microorganisms cannot survive prolonged exposure to UVC light, utilization of concentrated UVC in a closed environment such as a water holding tank or duct system has been shown to be lethal to many types of microorganisms including bacteria, viruses and fungi.

The present invention comprises a device and a technique for using the device, which can utilize UVC light to sterilize an intravenous long-dwelling catheter. The device and technique of the present invention eliminates many of the problems and limitations of conventional sterilization techniques and better enables health care professions to focus on treating the underlying medical condition without having to worry about the onset of a secondary, catheter-related infection.

In one embodiment of the present invention, an intravenous port is provided. The port features a catheter, an access head, a power source and a transmitter electrically connected to the power source. The catheter has proximal and distal ends and a lumen area extending longitudinally within the catheter. The distal end of the catheter is suitable for being inserted into a blood vessel. The access head is fastened to the proximal end of the catheter and features a signal reception means, such as for example an antenna, which is electrically connected to an ultraviolet (UV) light source such as for example a UV light emitting diode (LED). The light source is suitable for directing UV light of a predetermined wavelength into the lumen area of the catheter in a direction of the distal end. The transmitter of this embodiment is suitable for transmitting electrical signals of a predetermined frequency from the power source to the signal reception means of the access head in order to power the light source.

In one embodiment, the catheter and access head can be implanted into a patient in an area proximate one of the patient's central blood vessels and the transmitter and power source are mounted on an electric transmitting patch (ETP). The ETP can be affixed to the exterior surface of a patient in an area directly adjacent to or substantially proximate the implanted access head. When arranged in this fashion, the transmitter can wirelessly transmit electrical signals from the power source to the signal reception means of the implanted access head via electromagnetic induction through the patient's epithelial tissue. The reception means receives the electrical signals which are then transferred to the light source in order to power the light source.

The power source of the present invention can be any electrical energy source such as for example at least one battery cell or an alternating current (“AC”) power supply. Battery cells can be mounted on the same unit as the transmitter such as for example an ETP, or alternatively can be located on a separate component and electrically connected to the transmitter or ETP via wiring. When an AC power supply serves as the power source, an electrical wire having a plug and possibly an AC adaptor can be utilized in order to transmit electrical current from a wall outlet to the transmitter.

Another embodiment of the present invention is directed to a method of sterilizing an intravenous port via a UV light source, such as for example an UV light emitting diode (LED). In this embodiment, the method includes administering a sterile solution into the port such that the solution is directed into a lumen area of the catheter. An electrical signal is transmitted from a power source to a light source operatively associated with a catheter that is capable of being at least partially implanted into a patient proximate one of the patient's central blood vessels. The electrical signal powers the light source which emits UV light of a predetermined wavelength into a lumen area of the catheter. In this embodiment, the UV light is suitable for incapacitating microorganisms residing in the catheter.

In one embodiment of the present invention the device and technique have a UV source that is implanted within the patient that is powered wirelessly from outside the patient, for example by electromagnetic induction, so that a patient need not be constantly attached to an external UV source or power source.

Features and advantages of the present invention will be apparent from the following description and the appended claims when taken in conjunction with the accompanying drawings.

### BRIEF DESCRIPTION OF THE DRAWINGS

FIG. 1 is a perspective view of an intravenous port according to one embodiment of the present invention.

FIG. 2 is a cross section view of the access head and proximal end of the catheter of an intravenous port according to one embodiment of the present invention.

FIG. 3 is an exploded view of the access head of the intravenous port according to one embodiment of the present invention.

FIG. 4 is a schematic representation of the power source, wireless transmitter and access head of one embodiment of the present invention.

FIG. 5 is perspective view of the electric transmitting patch (ETP) according to one embodiment of the present invention.

FIG. 6 is a front elevational view of a human torso having an ETP affixed according to one embodiment of the present invention.

### DETAILED DESCRIPTION OF THE ILLUSTRATIVE EMBODIMENT

While the present invention is susceptible of embodiment in various forms, there is shown in the drawings a number of presently preferred embodiments that are discussed in greater detail hereafter. It should be understood that the present disclosure is to be considered as an exemplification of the present invention, and is not intended to limit the invention to the specific embodiments illustrated. It should be further understood that the title of this section of this application (“Detailed Description of the Illustrative Embodiment”) relates to a requirement of the United States Patent Office, and should not be found to limit the subject matter disclosed herein.

In this disclosure, the use of the disjunctive is intended to include the conjunctive. The use of the definite article or indefinite article is not intended to indicate cardinality. In particular, a reference to “the” object or “a” object is intended to denote also one of a possible plurality of such objects.

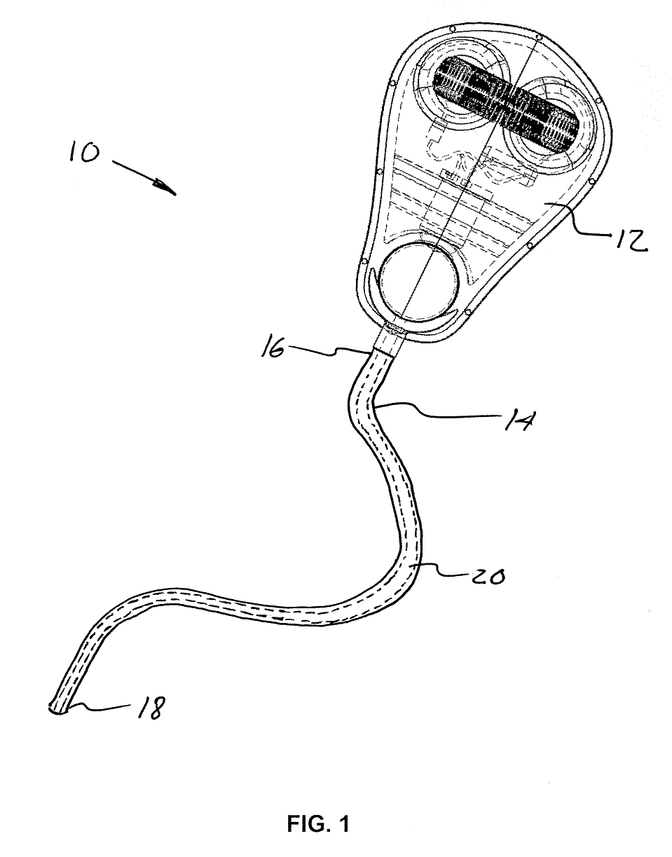

Referring now to the figures, and particularly to FIGS. 1-3, an intravenous port 10 according to one embodiment of the present invention is shown. As illustrated in FIG. 1, the port 10 features an access head 12 and catheter 14, having a proximal end 16 and a distal end 18. In use, it is contemplated that port 10 will be surgically implanted into the body of a patient in proximity to a central blood vessel, such as for example the jugular, subclavian or femoral vein, in a manner in which the distal end 18 of the catheter 14 is inserted into a blood vessel in a manner well known to medical practitioners. While port 10 of the present invention is intended for use with human beings, those having ordinary skill in the art will understand that all embodiments of the present invention can be used in connection with both humans and animals without departing from the novel scope of the present invention.

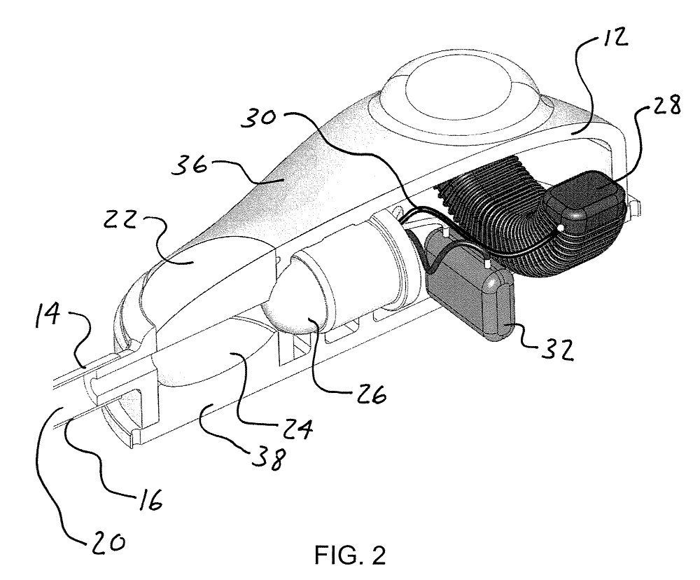

As shown in FIG. 2, the catheter 14 features a lumen 20 extending longitudinally within the catheter between the proximal end 16 and distal end 18. In this embodiment, it is contemplated that the lumen 20 will serve as a passageway for delivering fluids, including medicines and other necessary or desirable elements, from the access head 12 into the associated blood vessel such that they may be circulated throughout the patient's body. The catheter 14 can be comprised of a standard medical grade silicone and/or other materials as is known to persons having ordinary skill in the art.

In the embodiment illustrated in FIG. 2, the port 10 features an access head 12 fastened, or otherwise attached, to the proximal end 16 of the catheter 14. The access head 12 comprises a septum 22 and fluid well 24. The septum 22 acts as a barrier between the fluid well 24 and the exterior wall of the access head 12. It is contemplated that a syringe and hollow needle or cannula can be inserted through the septum 22 in order to administer fluids, including medications, to the patient. The septum 22 is comprised of a material capable of being pieced by a needle such that the fluid can be introduced into the fluid well 24 under pressure from a syringe. After insertion of the needle through the septum 22 and depressing the plunger of the syringe, the fluid will be forced through the lumen 20 of the catheter 14 and delivered to the patient through the associated blood vessel at the distal end 18 of the catheter 14.

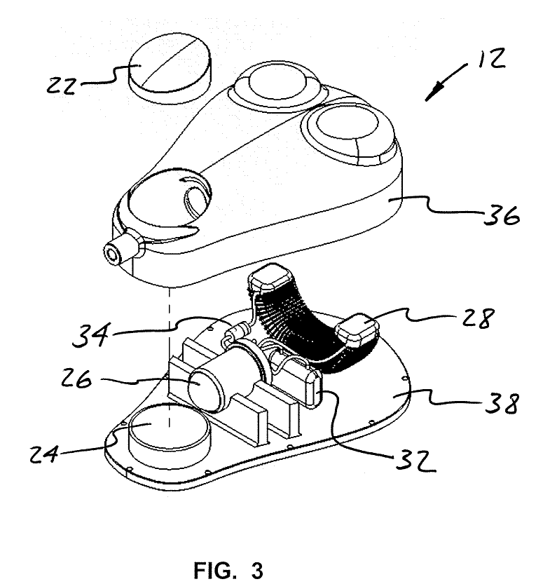

As shown in FIGS. 2-3, the access head 12 of port 10 can be comprised of an outer shell 36 and base 38, formed of an FDA approved medical grade silicon, or other appropriate material, which is suited to be surgically implanted into a patient. When implanted into a patient's body, the access head 12 will preferably be located just beneath the patient's epithelial tissue using the same methods practiced when implanting a standard Port-a-cath®, Pic or central line, in a manner well known to medical professionals. When the access head 12 is in this position, a healthcare professional will be able to easily locate the septum 22, which will be close enough to the surface of the patient's skin to allow for a needle to penetrate. Although it is preferable for the access head 12 be surgically implanted into the body of a patient, those having ordinary skill in the art will understand that an implantation outside the body of the patient can be made without departing from the novel scope of the present invention.

In the embodiment of the present invention shown at FIGS. 2-3, the access head 12 further features signal reception means 28, such as for example an antenna or other communications means, electrically connected to an ultraviolet (UV) light source 26. In one embodiment, the light source 26 is a UV light emitting diode (LED) suitable for directing UV light of a predetermined wavelength into the lumen 20 of the catheter 14; preferably directed towards the lumen's distal end 18. The reception means 28 is adapted to receive electrical signals of a predetermined frequency transmitted from a nearby transmitter (not shown) used to active the light source 26. As shown in FIGS. 2 and 3, the reception means 28 can be comprised of a helically wound coil and can be connected to the light source 26 through electrical circuitry 30 including a rectifier 32 and a Z-diode 34. Persons having ordinary skill in the art will recognize that there are any number of ways to provide connectivity between a communications means, such as an antenna or other radiator and a suitable light source, without departing from the novel scope of the present invention.

When the light source 26 is activated it will emit UV light of a predetermined wavelength (specifically a wavelength known to be lethal to microorganisms) through the fluid well 24 and into the lumen 20, at the proximal end 16 of the catheter 14. The specific wavelength of UV light is transmitted through the lumen 20 acting as a germicidal irradiant that sterilizes the inside of the catheter 15 by destroying microorganisms. The wavelength of the UV light emitted by the light source 26 is preferably between 100-280 nanometers (nm) and is thus in the range of UV light generally referred to as UV-C. The transmission of the UV-C light inside the fluid well 24 and lumen 20 is preferably to be accompanied by a sterile solution, such as saline, which is injected into the port 10 through the septum 22 during the sterilization procedure. It will be understood that the sterile solution assists in transmitting the UV light through the catheter 14 and additionally makes microorganisms within the lumen 20 more susceptible to UV exposure by separating them from the lumen wall and holding them in a suspended state. The UV light is transmitted through the sterile solution in the fluid well 24 and into the lumen 20 at the proximal end 16 of the catheter 14. In a preferred embodiment of the method of the present invention, the UV light is then directed through the lumen 20 towards the distal end 18 of the catheter 14. As the UV light is transmitted through the lumen 20, any microbes residing within the lumen 20 will be exposed to the light and will ultimately be destroyed.

While the amount of time of exposure to the UV light needed to sterilize the port 10 may vary, depending on a variety of factors including the intensity of the light source 26 and the size and configuration of the catheter 14 and others, sterilization of a long-dwelling catheter 14 has been determined to be most effective if the procedure is conducted over the course of several hours. Exposure to UV-C light within the close confines of the lumen 20 for that duration will deactivate the DNA of any microorganisms residing in the lumen 20 and thus destroy their ability to multiply and cause disease. The sterilization process can be repeated, as necessary if the port 10 is to be used for an extended period of time. The UV sterilization process of the present invention is a non invasive procedure that does not require constant manual attention from health care professionals; thereby providing an effective and comparatively inexpensive means to maintain a clean catheter and port.

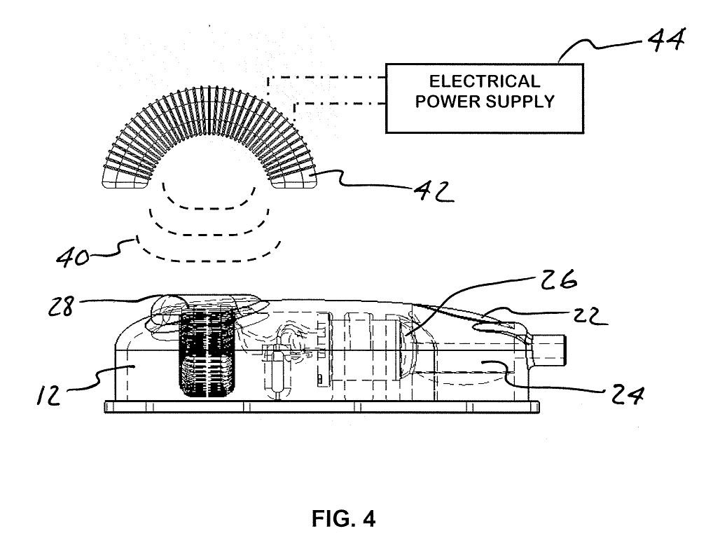

As shown in FIG. 4, the reception means 28, in one embodiment, can be an antenna adapted to receive wireless electrical signals 40 to activate a UV light source. Referring to FIG. 4, it will be seen that the present embodiment includes a wireless transmitter 42 maintained nearby to antenna 28, the transmitter 42 being connected to a power source 44. When the access head 12 is implanted inside the body of a patient, such wireless signals 40 can be transmitted, for instance via electromagnetic induction or other means as known by persons having ordinary skill in the art, through the patient's epithelial tissue. The power source 44 of the present invention can be any electrical energy source such as, for example, at least one battery cell or an alternating current (“AC”) power supply. Battery cells can be mounted on the same unit as the transmitter such as for example an electrical transmission patch (ETP), or alternatively can be located on a separate component and electrically connected to the transmitter or ETP via wiring or other electrical connection means as known by persons having skill in the art. When an AC power supply serves as the power source, an electrical wire having a plug and possibly an AC adaptor can be utilized in order to transmit electrical current from a wall outlet, or electrical generation means, to the transmitter.

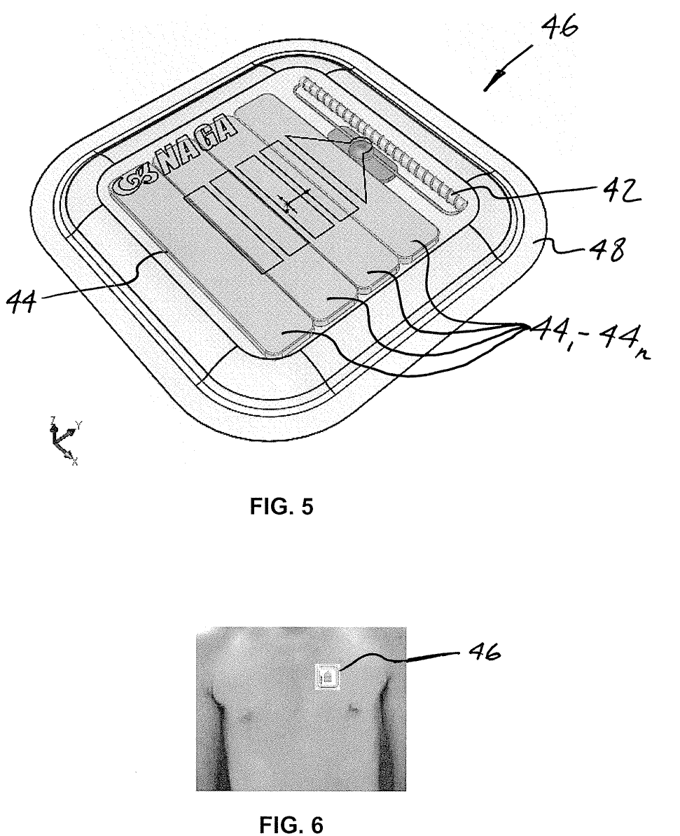

Referring to FIGS. 5 and 6 another embodiment of the present invention is illustrated therein. In this embodiment, the wireless transmitter 42 and the power source 44 are affixed to an electric transmitting patch (ETP) 46. The ETP 46 is adapted to be fastened to the exterior surface of a patent in an area substantially proximate to the implanted access head 12. In the embodiment illustrated in FIG. 5, the power source 44 is a plurality of battery cells 44₁-44ₙ. The battery cells 44₁-44ₙ generate electrical signals that are transmitted by the wireless transmitter 42. In the same manner as illustrated in FIG. 4, the electrical signals 40 are transmitted through the patient's epithelial tissue via electromagnetic induction and captured by the antenna of the implanted access head 12. The ETP 46 illustrated in FIGS. 5 and 6 features an adhesive area 48 around its perimeter that is suitable for the fastening of the ETP 46 to the skin of the patent.

When the ETP 46 is used in connection with the port 10, it has been determined that the best results are achieved when the ETP 46 is affixed to the patient in a location that is directly adjacent to or substantially proximate to the implanted access head 12. In such arrangements, the electric signals 40, transmitted by the wireless transmitter 42, have only a short distance to travel before being received by the antenna 28. As the signals may tend to dissipate as they travel away from their source through the patient's epithelial tissue, it will be understood that close proximity of the ETP to the access head 12 will be most effective.

FIG. 6 illustrates the ETP 46 of one embodiment affixed to the upper left quadrant of a human torso. In this position, the ETP 46 is suitable for use with a port 10 that has been implanted proximate to the subclavian vein. Although not pictured, it is contemplated that the access head 12 is positioned immediately behind the ETP 46. In this embodiment, the ETP 46 can be attached and removed from the patient without having to connect or disconnect any wiring from the patient. The ETP may further be made either for single use, and thus be disposable, or may be suitable for repeated use with a power source 44 that is replaceable or rechargeable.

The present invention is further directed to a method of sterilizing an intravenous port. Referring to FIGS. 1-2, in this method, a sterile solution is administered into the port 10 such that the solution is directed into a lumen 20 of the catheter 14. The method further comprises transmitting an electrical signal from a power source 44 to an ultraviolet (UV) light source 26, such as for example a light emitting diode (LED), which is operatively associated with a catheter 14, which is suitable for being at least partially implanted into a patient proximate to a central blood vessel. The electrical signals are then transferred to the light source 26 that is powered such that it emits UV light of a predetermined wavelength into the lumen area 20 of the catheter 14. The UV light is transmitted through the solution in a manner that is capable of incapacitating microorganisms residing in the catheter.

The solution, of the present example, can be administered into the port 10 by means of a syringe having a needle or cannula. In administering the solution in this manner, a syringe having a plunger member and needle or cannula is provided. The needle or cannula is driven into the port 10 through the septum 22 of the access head 12. After the needle is inserted into the access head 12, the plunger member of the syringe is pushed so that the solution contained within the syringe is driven from the syringe into the access head. Under pressure from the syringe, the fluid exits the access head 12 at the proximal end 16 of the catheter 14 and enters the lumen 20 of the catheter 14.

In another embodiment, the step of transmitting the electrical signal from a power source 44 to the light source 26 in the above described method is accomplished in part via electromagnetic induction through the patient's epithelial tissue. In this embodiment, an electrical signal is wirelessly transmitted from a transmitter located outside the patient's body to a signal reception means 28 positioned on an access head 12 implanted into the patient. The signals are then transmitted through associated electrical circuitry 30 from the reception means 28 to the light source 26. The light source 26 is sufficiently powered by the electrical signal to emit UV light of a predetermined wavelength into the lumen area 20 of the catheter 14. The UV light is transmitted through the solution in a manner that is capable of incapacitating microorganisms residing in the catheter.

The specific embodiments of the present invention have been described for the purpose of illustrating the manner in which the invention is made or used. It should be understood that the implementation of other variations and modifications of the invention and its various aspects will be apparent to one skilled in the art, and that the invention is not limited by the specific embodiments described. Therefore, it is contemplated to cover the present invention any and all modifications, variations, or equivalents that fall within the true spirit and scope of the basic underlying principles disclosed and claimed herein.

What is claimed is:

1. An intravenous port comprising:
a catheter having a proximal end and a distal end and a lumen extending longitudinally within the catheter between the proximal end and the distal end, the distal end of the catheter being suitable for insertion into a blood vessel;
an ultraviolet (UV) light source suitable for directing UV light of a predetermined wavelength through the lumen of the catheter from the proximal end to the distal end;
an access head fastened to the proximal end of the catheter, the access head having signal reception means connected to the ultraviolet (UV) light source;
a power source; and
a transmitter electrically connected to the power source, the transmitter being suitable for transmitting electrical signals from the power source to the signal reception means of the access head so as to activate the UV light source.

2. The intravenous port of claim 1 wherein the ultraviolet light source is a light emitting diode (LED).

3. The intravenous port of claim 1 wherein the catheter and access head are suitable for being implanted within a patient in an area proximate to a central blood vessel.

4. The intravenous port of claim 1 wherein the power source is at least one battery cell.

5. The intravenous port of claim 1 wherein the power source is an alternating current power supply.

6. The intravenous port of claim 1 wherein the light source emits UV light having a wavelength of between 100 nm and 280 nm.

7. The intravenous port of claim 3 wherein the transmitter is a wireless transmitter suitable and the reception means is an antenna, wherein the transmitter transmits wireless electrical signals from the power source to the antenna of the access head.

8. The intravenous port of claim 7 wherein the transmitter and power source are affixed to an electric transmitting patch (ETP) operatively associated with the access head and suitable for being affixed to an exterior surface of a patient in an area substantially proximate the implanted access head.

9. The intravenous port of claim 7 wherein the wireless electrical signals are transmitted from the wireless transmitter to the antenna via electromagnetic induction through the patient's epithelial tissue.

10. An intravenous port comprising:
a catheter having a proximal end and a distal end and a lumen area extending longitudinally within the catheter between the proximal end and the distal end, the distal end of the catheter being suitable for insertion into a blood vessel;
an access head fastened to the proximal end of the catheter, the access head having an signal reception means connected to a light emitting diode (LED), the LED suitable for directing ultraviolet light into the lumen area of the catheter in a direction of the distal end;
the catheter and access head suitable for being implanted within a patient in an area proximate a central blood vessel; and
an electric transmitting patch (ETP) operatively associated with the access head and suitable for being affixed to an exterior surface of the patient in an area substantially proximate the implanted access head, the ETP having a power source electrically connected to a wireless transmitter, the transmitter suitable for wirelessly transmitting electrical signals from the power source to the reception means of the implanted access head via electromagnetic induction through the patient's epithelial tissue.

11. The intravenous port of claim 10 wherein the power source is at least one battery cell.

12. The intravenous port of claim 10 wherein the power source is an alternating current power supply.

13. The intravenous port of claim 10 wherein the LED emits UV light having a wavelength between 100 nm and 280 nm.

14. The intravenous port of claim 10 wherein the reception means is an antenna.

15. A method of sterilizing an intravenous port comprising:
administering a sterile solution into the port such that the solution is directed into a lumen area of a catheter;
transmitting an electrical signal from a power source to an ultraviolet (UV) light source operatively associated with a catheter at least partially implanted into a patient proximate a central blood vessel;
powering the light source; and
emitting UV light of a predetermined wavelength into the lumen area of the catheter so that the UV light is transmitted through the solution in a manner that is capable of incapacitating microorganisms residing in the catheter.

16. The method of claim 15 wherein the step of transmitting an electrical signal from the power source to the UV light source comprises transmitting electrical current from a power source to the transmitter; transmitting a wireless electrical signal from a transmitter to a signal reception means of an access head housing the light source; receiving the wireless electrical signal; and transmitting the electrical signal from the reception means to the light source

17. The method of claim 15 wherein the step of administering the solution into the port comprises: providing a syringe having a needle; inserting the needle into an access head of the port; and pushing a plunger member of the syringe such that the solution is driven from the syringe into the access head.

18. The method of claim 16 wherein the step of transmitting a wireless electrical signal from the transmitter to the reception means occurs via electromagnetic induction through the patient's epithelial tissue.

19. A method of sterilizing an intravenous port having a catheter, the method comprising:
administering a sterile solution into the port such that the solution is directed into a lumen area of the catheter;
transmitting a wireless electrical signal from a power source to an antenna of an access head implanted into a patient, the wireless transmission occurring via electromagnetic induction through the patient's epithelial tissue;
transferring the wireless signal from the antenna of the access head to an ultraviolet (UV) light emitting diode (LED) operatively associated with the catheter at least partially implanted into the patient in a location proximate a central blood vessel;
powering the LED; and
emitting UV light of a predetermined wavelength into the lumen area of the catheter so that the UV light is transmitted through the solution in a manner that is capable of incapacitating microorganisms residing in the catheter.

20. The method of claim 19 wherein the step of administering the solution into the port comprises: providing a syringe having a needle; driving the needle into an access head of the port; and pushing a plunger member of the syringe such that the solution is driven from the syringe into the access head.
